(19) United States
(12) Patent Application Publication
(10) Pub. No.: US 2002/0026211 A1
(43) Pub. Date: Feb. 28, 2002

(54) Vascular device having emboli and thrombus removal element and methods of use

(76) Inventors: Farhad Khosravi, San Mateo, CA (US); Amr Salahieh, Campbell, CA (US); Jeff A. Krolik, Campbell, CA (US); Stephen Ramee, New Orleans, LA (US)

Correspondence Address: FISH & NEAVE, 1251 AVENUE OF THE AMERICAS, 50TH FLOOR, NEW YORK, NY, 10020-1105, US

(21) Appl. No.: 09/764,777

(22) Filed: Jan. 16, 2001

Related U.S. Application Data
(63) Continuation-in-part of application No. 09/470,681, filed on Dec. 23, 1999.

(51) Int. Cl. A61M 29/00

(52) U.S. Cl. 606/200

(57) ABSTRACT

Apparatus and methods are provided for use in filtering emboli or removing thrombus from a vessel, wherein a vascular device comprises a vascular filter for capturing emboli and optionally, a thrombectomy element for excising or ablating thrombus. The vascular filter comprises a support hoop having one or more articulation regions connected near a distal end of a guide wire, and a blood permeable sac affixed to the support hoop so that the support hoop forms a mouth of the blood permeable sac. In a preferred embodiment, the thrombectomy element also comprises a vascular filter support hoop, and may include filter frame, locking feature or tensioning thread.

### REFERENCE TO RELATED APPLICATIONS

This application is a continuation-in-part of U.S. Pat. application Ser. No. 09/470,681, filed Dec. 23, 1999, which is a continuation-in-part of U.S. Pat. application Ser. No. 09/364,064 filed Jul. 30, 1999.

### FIELD OF THE INVENTION

The present invention relates to apparatus and methods for removing thrombus from within a vascular system and filtering emboli within a vascular system. More particularly, the present invention provides a low profile self-expanding vascular device useful for capturing emboli, and which may include a thrombectomy element for the removal of thrombus.

### BACKGROUND OF THE INVENTION

Many percutaneous procedures for treating vascular disease have been proposed. However, these procedures often dislodge material from the vessel walls. This dislodged material, known as emboli, enters the bloodstream, and may be large enough to occlude smaller downstream vessels, potentially blocking blood flow to tissue. The resulting ischemia poses a serious threat to the health or life of a patient if the blockage occurs in critical tissue, such as the heart, lungs, or brain.

Numerous previously known methods and apparatus have been proposed to reduce complications associated with embolism or release of thrombus. U.S. Pat. No. 5,833,644 to Zadno-Azizi et al., for example, describes the use of a balloon-tipped catheter to temporarily occlude flow through a vessel from which a stenosis is to be removed. Stenotic material removed during a treatment procedure is evacuated from the vessel before the flow of blood is restored. A drawback of such previously known systems, however, is that occlusion of antegrade flow through the vessel may result in damage to the tissue normally fed by the blocked vessel.

U.S. Pat. No. 5,814,064 to Daniel et al. describes an emboli filter system having a radially expandable mesh filter disposed on the distal end of a guide wire. The filter is deployed distal to a region of stenosis, and any interventional devices, such as angioplasty balloons or stent delivery systems, are advanced along the guide wire. The filter is designed to capture emboli generated during treatment of the stenosis while permitting blood to flow through the filter. Similar filter systems are described in U.S. Pat. No. 4,723,549 to Wholey et al. and U.S. Pat. No. 5,827,324 to Cassell et al.

One disadvantage of radially expandable filter systems such as described in the foregoing patents is the relative complexity of the devices, which typically comprise numerous parts. Connecting more than a minimal number of such parts to a guide wire generally increases delivery complications. The ability of the guide wire to negotiate tortuous anatomy is reduced, and the profile of the device in its delivery configuration increases. Consequently, it may be difficult or impossible to use such devices in small diameter vessels, such as are commonly found in the carotid artery and cerebral vasculature. Moreover, such filter devices are generally incapable of preventing material from escaping from the filter during the process of collapsing the filter for removal.

International Publication No. WO 98/39053 describes a filter system comprising an elongated member, a radially expandable hoop and a cone-shaped basket. The hoop is affixed to the elongated member, and the cone-shaped basket is attached to the hoop and the elongated member, so that the hoop forms the mouth of the basket. The filter system includes a specially configured delivery catheter that retains the mouth of the basket in a radially retracted position during delivery.

While the filter system described in the foregoing International Publication reduces the number of components used to deploy the cone-shaped basket, as compared to the radial strut-type filter elements described hereinabove, it too has drawbacks. Chief among these, it is expected that it will be difficult to reduce the diameter of the radially expandable hoop to its retracted position. In particular, as the hoop is contracted through smaller radii of curvature, the stiffness of the hoop is expected to increase dramatically. This increased stiffness prevents the hoop from being contracted more tightly, and is expected to result in a delivery profile too large to permit use of the device in critical regions of the body, such as the smaller coronary arteries, carotid arteries, and cerebral vasculature.

In view of the foregoing disadvantages of previously known apparatus and methods, it would be desirable to provide a vascular device for filtering emboli from a vascular system that overcomes such disadvantages of previous vascular filters.

It also would be desirable to provide a vascular device for removing thrombus from a vascular system that overcomes the disadvantages of previously known thrombectomy devices.

It also would be desirable to provide a vascular device that is capable of being contracted to a small delivery profile, thus permitting use of the device in small vessels.

It further would be desirable to provide a vascular device that is capable of being contracted to a sufficiently small profile that it may be retrieved using the guide wire lumen of previously known treatment devices, and without the need for specialized delivery catheters.

It still further would be desirable to provide a vascular device that reduces the risk of emboli from escaping from the device when the device is collapsed and removed.

### SUMMARY OF THE INVENTION

In view of the foregoing, it is an object of the present invention to provide a vascular device that overcomes disadvantages of previously known thrombectomy/embolectomy devices and vascular filters, and employs few components.

It also is an object of this invention to provide a vascular device that is capable of being contracted to a small delivery profile, thus permitting use of the device in small vessels.

It is a further object of the present invention to provide a vascular device that is capable of being contracted to a sufficiently small profile that it may be retrieved using the guide wire lumen of previously known treatment devices, and without the need for specialized delivery catheters.

It is another object of this invention to provide a vascular device that reduces the risk of emboli or thrombus removed from the vessel wall escaping from the device when the device is collapsed and removed.

These and other objects of the present invention are accomplished by providing a vascular device comprising a blood permeable sac affixed at its perimeter to a support hoop. The support hoop is attached in a distal region of an elongated member, such as a guide wire, and supports a proximally-oriented mouth of the sac when the filter is deployed in a vessel. In accordance with the principles of the present invention, the support hoop includes one or more reduced-thickness articulation regions that enable the support hoop to be contracted to very small radii of curvature without the problems of increased stiffness and kinking of previously known filters.

The support hoop preferably has a curved profile that prevents the one or more articulation regions, when folded, from damaging the wall of the vessel. This feature also permits the device to effectively contact the walls of the vessel and reduce emboli and/or thrombus removed from the vessel wall from bypassing the sac. When the vascular device is collapsed into a sheath for removal, the articulation region causes the sides of the support hoop to fold inwards towards one-another. This in turn closes the mouth of the sac and reduces the potential for captured emboli or thrombus to be released from the vascular device during removal.

Advantageously, use of one or more articulation regions permits the vascular device of the present invention to be contracted to very small diameters, thereby enabling the use of delivery catheters having diameters as small as 2.5 Fr. Moreover, the vascular device of the present invention may be retracted within the guide wire lumen of conventional treatment devices, such as angioplasty catheters and stent delivery systems, thereby obviating the need to re-insert a specialized delivery catheter to remove the vascular device. Alternatively, specialized delivery apparatus may be provided.

In accordance with one aspect of the present invention, the vascular device also may include a thrombectomy element comprising a blood permeable sac and support hoop as described above. Alternatively, the sac and hoop may be provided as a vascular filter used in conjunction with the thrombectomy element. In such embodiments, the thrombectomy element preferably is coupled to the elongated member proximal of the vascular filter, or may comprise a separate catheter. In a preferred embodiment, the thrombectomy element is similar in construction to the vascular filter, and may be retracted independently. Alternatively, the thrombectomy element may be any conventional atherectomy device used in conjunction with the vascular filter and may be advanced and retracted either in conjunction with, or independently of, the vascular filter.

Methods of using the vascular device of the present invention are provided.

### Brief Description Of The Drawings

The above and other objects and advantages of the present invention will be apparent upon consideration of the following detailed description, taken in conjunction with the accompanying drawings, in which like reference characters refer to like parts throughout, and in which:

FIGS. 1A and 1B are, respectively, a side sectional of a previously known vascular filter contracted within a delivery sheath and an end view of that vascular device deployed in a vessel;

FIGS. 2A and 2B are, respectively, a perspective view of a vascular filter constructed in accordance with the principles of the present invention in a deployed state, and a detailed view of the articulation region of the filter of FIG. 2A;

FIG. 3 is a perspective view of the vascular filter of FIGS. 2 in a folded configuration, prior to removal;

FIG. 4 is a plan view of the vascular filter of FIGS. 2;

FIGS. 5A and 5B are, respectively, side-sectional views depicting a vascular device, including a thrombectomy element, disposed within a delivery sheath, and in a deployed state;

FIGS. 6A-6E are side-sectional views depicting a method of deploying, using and retrieving the vascular device of FIGS. 5;

FIGS. 7A and 7B are, respectively, side-sectional views depicting an alternative embodiment of the vascular device of FIGS. 5 disposed within a delivery sheath, and in the deployed state;

FIGS. 8A and 8B are views depicting another alternative embodiment of the present invention having multiple articulation regions and shown, respectively, in side-view in the deployed state and in side-view, partially in section, disposed within a delivery sheath;

FIG. 9 is a side-view of an alternative embodiment of the vascular device of FIG. 8 comprising a positive locking feature;

FIG. 10 is a side-view depicting yet another alternative embodiment of the vascular device of the present invention having a filter frame;

FIG. 11A-11D are, respectively, isometric, bottom, side, and front views of the filter frame of FIG. 10; and

FIG. 12 is a side-view of an alternative embodiment of the vascular device of FIG. 10 having a tension thread.

### DETAILED DESCRIPTION OF THE INVENTION

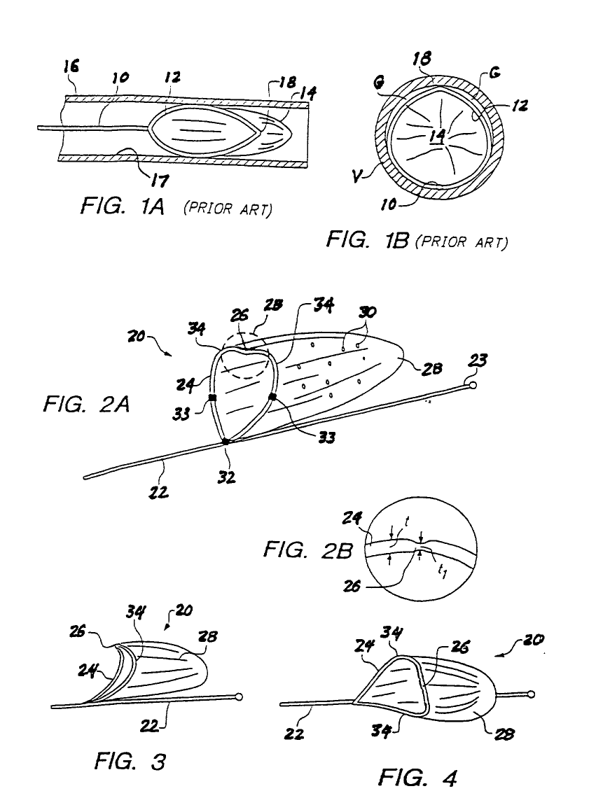

Referring to FIGS. 1A and 1B, some of the disadvantages associated with previously known vascular filters, such as the emboli filters described in the above-mentioned International Publication WO 98/39053, are described. The vascular filter comprises guide wire 10 having hoop 12 coupled to its end. Filter sac 14 is affixed to hoop 12, so that when delivery catheter 16 is retracted proximally and guide wire 10 is held stationary, hoop 12 radially expands to contact the walls of a vessel.

As described hereinabove, one difficulty with such vascular filters is that the hoop used to support the filter sac experiences increased stiffness when contracted to small diameters, i.e., due to the sharp directional change at the tip of the hoop, thereby limiting the minimum delivery profile achievable for such instruments. Although this effect may be reduced by decreasing the thickness of the wire employed in hoop 12, at the point at which the wire becomes sufficiently thin to accommodate the bending stresses, the wire is too thin to effectively radially expand and urge the filter sac into engagement with the vessel wall.

On the other hand, as shown in FIGS. 1A and 1B, the bending stresses imposed upon the hoop of such previously known devices, if drawn within a delivery catheter, may be sufficiently high to result in the formation of kink 18 at the tip of the hoop. This “kinking” effect becomes more severe in sheaths having a small inner diameter. Thus, for example, applicant has observed that when sheaths having inner diameters of 0.035″ or smaller are used, a hoop of nitinol or multi-strand nitinol cable having a diameter of 0.0055 inches will form kink 18. Kink 18 in turn may apply relatively high localized pressure and friction against wall 17 of sheath 16, thereby making the vascular filter difficult to deploy. In particular, the kink may impale wall 17 of delivery sheath 16 and may make it difficult or impossible to deploy the vascular filter, especially in tortuous anatomy.

In addition, when the filter is subsequently deployed in vessel V, as shown in FIG. 1B, kink 18 may deform the pre-formed shape of hoop 12, impairing the ability of the filter to seal against the walls of vessel V. This may in turn lead to the presence of gaps G between the perimeter of the hoop and the vessel wall, depending upon the severity of the kink. Consequently, emboli may pass through the gaps with antegrade flow and significantly reduce the efficacy of the filter. Additionally, kink 18 may be sufficiently sharp to damage or dissect the wall of vessel V when the filter is deployed.

The vascular filter of the present invention solves the above-described disadvantages, providing a vascular filter with a self-expanding support hoop that is sufficiently thick to radially expand and urge a blood permeable sac into engagement with the vessel wall, but which includes an articulation region that overcomes the problems associated with kinking. In particular, the vascular filter includes a reduced thickness articulation region and a pre-formed curved profile that avoids the difficulties of previously known systems while providing a high degree of efficacy in capturing emboli or thrombus, and ease of deployment and retrieval.

Referring now to FIGS. 2A and 2B, vascular filter 20 constructed in accordance with the principles of the present invention comprises guide wire 22, support hoop 24 having articulation region 26, and blood permeable sac 28 affixed to support hoop 24. Sac 28 is coupled to support hoop 24 so that the support hoop 24 forms an opening for the sac. Support hoop 24 preferably is connected to guide wire 22 near distal end 23 of the guide wire.

Sac 28 preferably is constructed of a thin, flexible biocompatible material, such as polyethylene, polypropylene, polyurethane, polyester, polyethylene tetraphlalate, nylon or polytetrafluoroethylene, or combinations thereof. The material should be sufficiently thin, such that the sac is nonthrombogenic. Sac 28 includes openings or pores 30 that permit blood cells to pass through the sac substantially unhindered, while capturing any larger emboli that may be released during a procedure such as angioplasty or stent placement. In a preferred embodiment, sac 28 has openings or pores 30 in a range of about 20 to 400 microns in diameter, and more preferably, about approximately 80 microns. These pores sizes will permit red blood cells (which have a diameter of approximately 5 microns) to easily pass through the sac.

Pores 30 are preferably formed by a laser drilling process. For example, a thin sheet of the flexible biocompatible material may be thermoformed to create sac 28, for example, by stretching the sheet over a mandrel, by dip forming, or by blow molding. Sac 28 may alternatively be fabricated from an extruded tube of the biocompatible material. A flat metal mask, with tiny holes approximately the size of pores 30, may then be placed in front of the sac. A laser having a beam diameter equal to or greater than the diameter of the material illuminates the mask. The laser beam passes through the holes in the mask and strikes the material, thereby forming pores 30 in sac 28.

Laser drilling also may be accomplished with a laser having a beam diameter approximately the size of pores 30, in which case pores 30 may be drilled individually. Sac 28 alternatively may comprise a woven material, for example, formed from the above-mentioned polymers, having a pore diameter determined as a function of the pattern and tightness of the weave.

Support hoop 24 comprises a hoop having a circular or rectangular cross-section that is formed of a super-elastic material, such as a nickel-titanium alloy (“nitinol”). During deployment and retrieval of vascular filter 20, described hereinafter, support hoop 24 folds in half and collapses to fit within a small diameter delivery sheath. When vascular filter 20 is in a deployed state, as depicted in FIG. 2A, support hoop 24 resumes its pre-formed shape. Support hoop 24 preferably comprises nitinol wire, although it also may be formed from a multi-strand nitinol cable, a spring tempered stainless steel, or other super-elastic material.

In accordance with the principles of the present invention, support hoop 24 includes one or more reduced-thickness articulation regions 26, and preformed curved regions 34. As depicted in FIG. 2B, articulation region 26 includes a region having reduced thickness t₁ compared to thickness t of the remainder of support hoop 24. Articulation region 26 and curved regions 34 enable support hoop 24 to fold with a predetermined shape when vascular filter 20 is collapsed to a contracted state for delivery or retrieval.

In FIG. 2B, articulation region 26 is depicted as a localized reduction in the thickness of support hoop 24, as may be achieved, for example, using conventional grinding, chemical etching, or electrodeless polishing processes. Alternatively, support hoop 24 may be continuously tapered along its circumference, so that articulation region 26 results from a more gradual reduction in the wall thickness of the support hoop. Tapering support hoop 24 may permit greater flexibility in the vicinity of articulation region 26, thus enabling support hoop 24 to fold more easily at the articulation region. Such tapering of the thickness of the support hoop along a portion of its circumference also may reduce the potential for stress-induced fracture typically associated with abrupt changes in diameter.

In a preferred embodiment of vascular filter 20 of the present invention, vascular filter 20 easily fits within a delivery sheath having an inner diameter of 0.033″, and, more preferably, may be used with a delivery sheath having an inner diameter as small as 0.026″. The deployed diameter of support hoop 24 preferably is approximately 7 mm, while guide wire 22 preferably has a diameter of 0.014″. The distal end of guide wire 22 also may be tipped with a spring section or coil tip, as is per se known.

Support hoop 24 preferably is constructed of 0.0055″ nitinol wire tapered (by a grinding, chemical etching, or electrodeless polishing process) to 0.0025″ at articulation region 26. Specifically, articulation region 26 preferably consists of a length about 0.05″ long and having a diameter of 0.0025″, coupled on either side to curved regions 34. Each of curved regions 34 includes a length of wire that is tapered from a diameter of 0.055″ to a diameter of 0.0025″ over a length of about 0.025″. Support hoop 24 also may include radiopaque features, such as gold or platinum bands 33, spaced at intervals around the circumference of support hoop 24, or a coil of radiopaque material wrapped around the support hoop, or a gold plated coating.

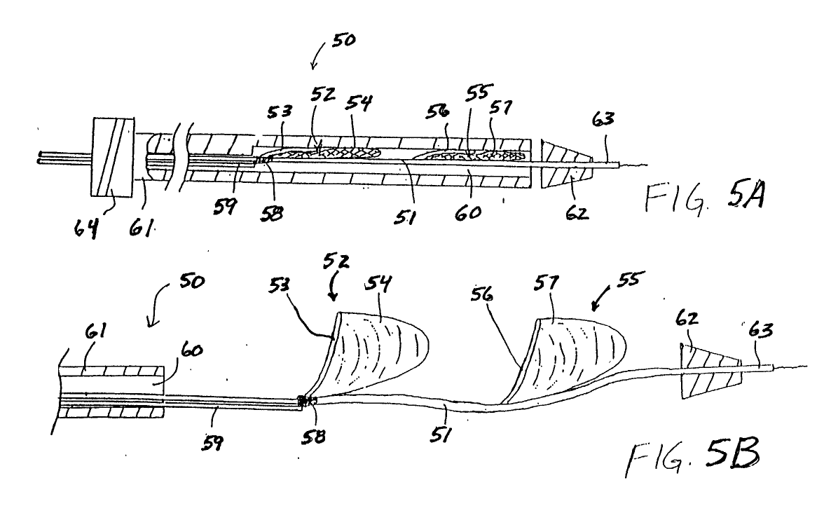

Referring to FIGS. 3 and 4, additional features of vascular filter 20 are described. FIG. 3 depicts vascular filter 20 of FIG. 2A in a contracted state, while FIG. 4 illustrates a directional change in support hoop 24 preferably caused by the presence of curved regions 34. Advantageously, use of articulation region 26 and the curved profile of support hoop 24 introduced by curved regions 34 also cause support hoop 24 to fold in half during retrieval. As shown in FIG. 3, support hoop 24 folds in half, effectively closing the mouth of blood permeable sac 28 and preventing the escape of collected emboli or thrombus. This feature also may permit the use of a smaller or shallower sac than would otherwise be possible, without increasing the risk of material escaping from the filter when the sac is collapsed for retrieval. Use of a smaller or shallower sac also enables vascular filter 20 to be delivered in a smaller delivery sheath, having an inner diameter as small as 0.026″ for the preferred embodiment.

Vascular devices of the present invention also may comprise a thrombectomy element constructed similarly to vascular filter 20 described above. Alternatively, vascular filter 20 may be used in conjunction with the thrombectomy element. In such embodiments, the thrombectomy element preferably is coupled to the elongated member proximal of the vascular filter, or may comprise a separate catheter. In a preferred embodiment, the thrombectomy element is similar in construction to filter 20, and may be retracted independently. Alternatively, the thrombectomy element may be any conventional atherectomy device used in conjunction with the vascular filter and may be advanced and retracted either in conjunction with, or independently of, the vascular filter.

Referring now to FIGS. 5A and 5B, an illustrative embodiment of a vascular device of the present invention including a thrombectomy element is described. Vascular device 50 comprises guide wire 51, thrombectomy element 52 including support hoop 53 and blood permeable sac 54, and vascular filter 55 including support hoop 56 and blood permeable sac 57. Filter hoop 56 is attached to guide wire 51 while thrombectomy hoop 53 is attached to ring 58. Ring 58 is attached to pull wire 59 and has a bore through which guide wire 51 passes. Ring 58 therefore acts as a linear bearing and allows thrombectomy hoop 53 to be moved by pull wire 59 independently of guide wire 51. Alternatively, thrombectomy element 52 may omit sac 54 and simply comprise a wire hoop; in this case severed thrombus is captured by vascular filter 55.

In FIG. 5A, support hoops 53 and 56 and blood permeable sacs 54 and 56 are contracted to a delivery state within lumen 60 of delivery sheath 61. Delivery sheath 61 includes nose cone 62 affixed to distal region 63 of guide wire 51. In FIG. 5B, vascular device 50 is shown deployed in a vessel. As illustrated in FIG. 5B, vascular filter 55 expands to engage the perimeter of the vessel and prevent thrombus from bypassing the blood permeable sac, while thrombectomy element 52 engages the vessel wall proximal of vascular filter 55. As described hereinbelow, proximal movement of thrombectomy device 52 scrapes thrombus from the wall of the vessel when pull wire 59 pulls ring 58 and support hoop 53 proximally.

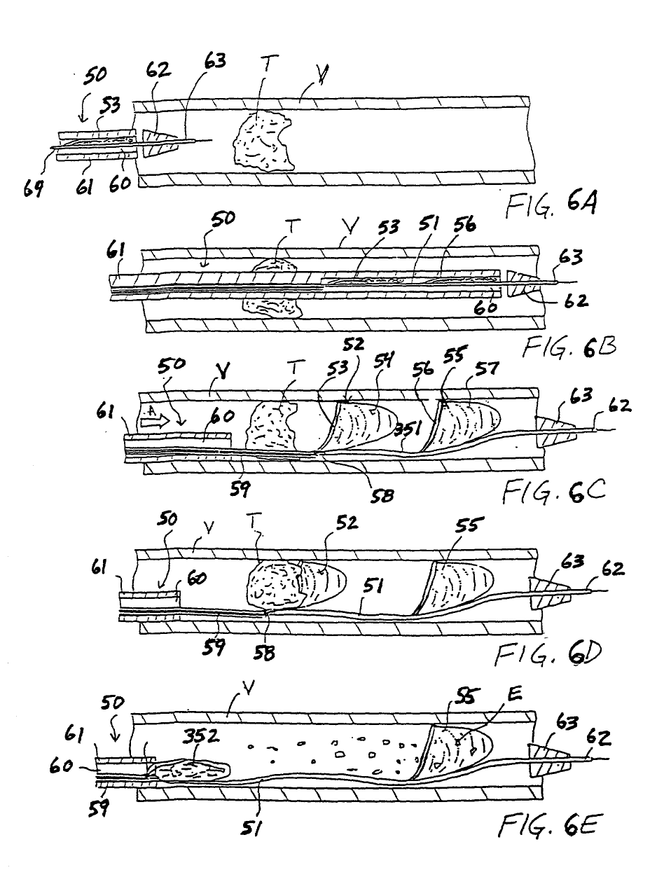

Referring now to FIGS. 6A-6E, an illustrative method of using the vascular device of FIGS. 5 for thrombectomy is described. In FIG. 6A, guide wire 51 is manipulated into position proximal to thrombus T within vessel V using well-known percutaneous techniques. Vascular device 50 of FIGS. 5A and 5B is disposed in its contracted delivery state within the distal end of delivery sheath 61 and the delivery sheath is advanced through the vessel using distal end 63 of guide wire 51. The sides of support hoops 53 and 56 are folded together and become elongated when drawn within delivery sheath 61, as described with respect to vascular device 20 of FIGS. 2-4.

With respect to FIG. 6B, once delivery sheath 61 is disposed at the desired location proximal to thrombus T within a patient's vessel V, such as a coronary artery or carotid artery, for example, based on the position of, for example, radiopaque bands under a fluoroscope, vascular device 50 is advanced through thrombus T. Distal end 63 of guide wire 51 is advanced through the lesion, then nose cone 62 gradually increases the diameter of the void within thrombus T so that the remainder of delivery sheath 61 can be advanced far enough that thrombectomy element 52 (still within delivery sheath 61) is located distal to thrombus T.

With vascular device 50 in position, guide wire 51 is held stationary while delivery sheath 61 is retracted proximally, as seen in FIG. 6C. Alternatively, delivery sheath 61 may be held stationary while guide wire 51 is advanced. In either case, when vascular device 50 is no longer confined within delivery sheath 61, support hoops 53 and 56 expand to seal against the walls of the vessel V and deploy blood permeable sacs 54 and 57, respectively. Blood continues to flow through vessel V in direction A, impeded only by thrombus T.

In FIG. 6D, once vascular device 50 is deployed in vessel V, thrombus T is removed in the following manner. Vascular filter support hoop 53 is rigidly attached to guide wire 51, while thrombectomy support hoop 53 is attached to pull wire 59 via ring 58. Thrombectomy element 52 then is retracted proximally to scrape along the wall of the vessel V by motion at the proximal end of pull wire 59. Thrombus T, located proximal to thrombectomy element 52, is excised so that it is captured in blood permeable sac 54 during the retraction.

With respect to FIG. 6E, once thrombus T has been captured within sac 54, pull wire 59 is pulled proximally to cause the sides of thrombectomy support hoop 53 to collapse together to close the mouth of sac 28 (see FIG. 3). Additional proximal retraction of pull wire 59 causes support hoop 53 and sac 54 to enter within lumen 60 of delivery sheath 61, restoring normal blood flow to vessel V. Meanwhile, vascular filter 55 is in a position distal to thrombectomy element 52 to trap emboli E, i.e., pieces of plaque dislodged from either thrombus T or the walls of vessel V by thrombectomy element 52. Once any emboli E have been collected, filter hoop 56 and sac 57 are retracted into delivery sheath 61 by motion at the proximal end of guide wire 51, in a manner similar to the retraction of hoop 53 and sac 54. Once guide wire 51 has been fully retracted and nose cone 62 at the distal end 63 of guide wire 51 is again in contact with delivery sheath 61, the delivery sheath is withdrawn with vascular device 50, the trapped thrombus T and any trapped emboli E.

Advantageously, the compliant design of vascular device 50 permits the device to be contracted to its delivery state within the guide wire lumen of conventional previously known interventional devices. Accordingly, unlike previously known vascular devices, which require removal of the interventional device followed by re-insertion of a specially designed catheter to retrieve the vascular device, the system of the present invention reduces the time, effort and trauma of this additional step. Instead, the vascular device may be readily closed and retrieved upon completion of the interventional procedure.

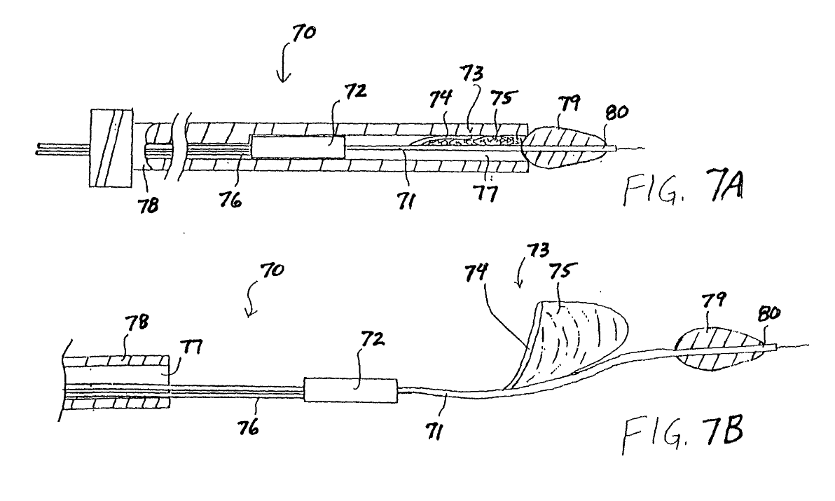

Referring now to FIGS. 7A and 7B, an alternative embodiment of the vascular device of FIG. 5 is described. Vascular device 70 comprises guide wire 71, thrombectomy element 72 and vascular filter 73 including support hoop 74 and blood permeable sac 75. Filter hoop 74 is attached to guide wire 71, while thrombectomy element 72 is disposed to slide along guide wire 71. Alternatively, thrombectomy element 72 may be disposed on a separate catheter element that extends either through lumen 77 of delivery sheath 78 or is separately disposed proximal of vascular filter 73.

FIG. 7A shows thrombectomy element 72 and vascular filter 73 contracted in a delivery state within lumen 77 of delivery sheath 78. Delivery sheath 78 includes nose cone 79 affixed to distal region 80 of guide wire 71. In FIG. 7B, vascular device 70 is shown in the deployed state. Thrombectomy element 72 may comprise any of a family of known thrombectomy, atherectomy, or, alternatively, drug delivery devices suitable for use in conjunction with device 73.

Specifically, thrombectomy element 72 may comprise any of: a rotary ablation device, such as described in U.S. Pat. Nos. 4,867,156 to Stack et al., 4,990,134 to Auth, and 5,314,407 to Auth et al.; an atherectomy technology, such as described in U.S. Pat. Nos. 5,181,920 to Mueller et al., and 5,074,841 to Ademovic et al.; or a balloon embolectomy technology, such as described in U.S. Pat. Nos. 3,923,065 to Nozick et al., 5,769,871 to Mers Kelly et al., 5,192,290 to Hilal, 5,112,347 to Taheri, and 4,030,503 to Clark III. All of the foregoing patents are incorporated herein by reference. Thrombectomy element 72 alternatively may comprise a wire loop or ring such as alternatively described for the embodiment of FIGS. 5A and 5B, a laser ablation device, a chemical flushing system, etc.

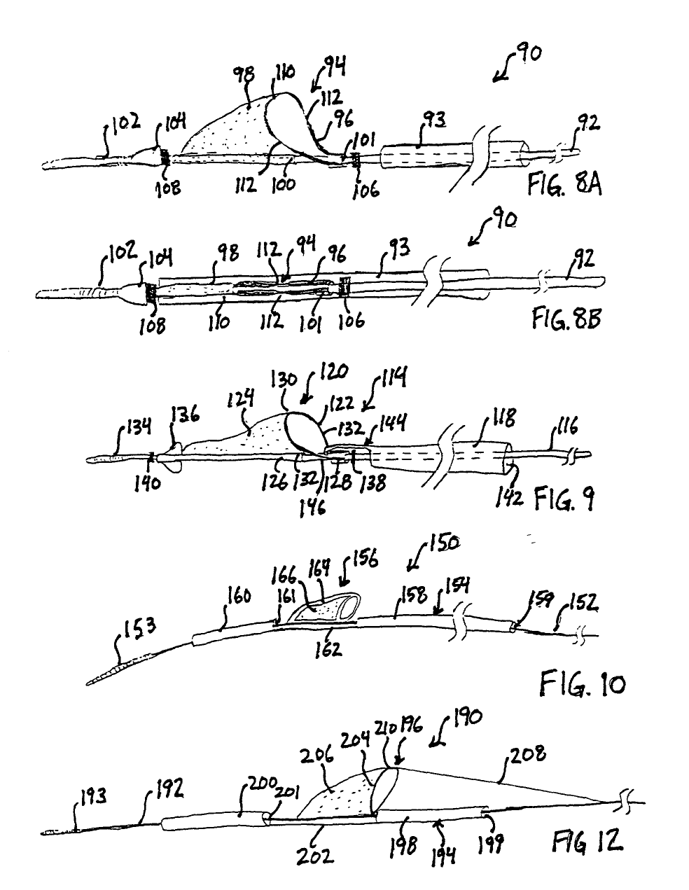

With reference to FIGS. 8A and 8B, another alternative embodiment of the present invention is described wherein the vascular filter element includes multiple articulation regions. Vascular device 90 comprises guide wire 92, sheath 93, and filter 94 comprising support hoop 96, blood permeable sac 98, spinner tube 100, and bearing 101. Sac 98 is attached along its length to spinner tube 100, which is coaxially and slidably disposed about guide wire 92. Support hoop 96 is attached to bearing 101, which is also coaxially and slidably disposed about guide wire 92. Guide wire 92 comprises floppy distal end 102, nose cone 104, proximal stop 106, and distal stop 108. Filter 94 is disposed between the proximal and distal stops.

Support hoop 96 is similar to support hoop 24 of FIG. 2A, except that it includes multiple articulation regions. Closure articulation region 110 facilitates collapse of filter 94 to the delivery configuration of FIG. 8B within sheath 93, while tracking articulation regions 112 act as bend points for easy tracking in tortuous anatomy. Tracking articulation regions 112 beneficially allow vascular device 90 to be used in a wider variety of applications, including neurothrombectomy applications.

Referring to FIG. 9, yet another alternative embodiment of the present invention having a positive locking feature is described. Vascular device 114 comprises guide wire 116, sheath 118, and filter 120 including support hoop 122, blood permeable sac 124, spinner tube 126, and bearing 128. Sac 124 is attached along its length to spinner tube 126, while support hoop 122 is attached to bearing 128. Both spinner tube 126 and bearing 128 are coaxially and slidably disposed about guide wire 116. Spinner tube 126 comprises nose cone 136, thereby allowing rotation of guide wire 116 without rotation of the larger diameter nose cone. Support hoop 122 comprises articulation region 130 disposed between curved regions 132. Guide wire 116 comprises floppy distal end 134, proximal stop 138, and distal stop 140, any of which may be radiopaque to facilitate positioning within a vessel. Filter 120 is disposed between the proximal and distal stops.

Sheath 118 comprises lumen 142, in which filter 120 is disposed in a collapsed configuration during delivery and retrieval. Sheath 118 further comprises locking distal end 144 having wedge 146. During deployment, sheath 118 is retracted with respect to filter 120 to allow support hoop 122 to expand and sealingly engage a patient's vessel. Further retraction of sheath 118 causes wedge 146 of distal end 144 to lodge between opposing curved regions 132 of support hoop 122, thereby inhibiting articulation of articulation region 130 and “locking” hoop 122 in the deployed configuration.

When used as thrombectomy elements in a dual arrangement as depicted in FIGS. 5, the support hoops of the present invention may have a tendency to close as they are retracted through thrombus in the manner seen, for example, in FIG. 6D. Referring again to FIG. 9, locking hoop 122 in the deployed configuration allows the hoop to be retracted through thrombus without closing the mouth of the hoop. Once thrombus has been captured in blood permeable sac 124, sheath 118 may be advanced with respect to hoop 122 in order to remove wedge 146 from between curved regions 132 of the support hoop, thereby “unlocking” the support hoop. Further advancement of sheath 118 with respect to hoop 122 causes filter 120 to articulate at articulation region 130 and collapse for retrieval within lumen 142 of sheath 118.

Referring to FIG. 10, a still further alternative embodiment of the present invention having a filter frame is described. As with vascular device 114 of FIG. 9, vascular device 150 of FIG. 10 addresses the potential for support hoops of the present invention to close as they are retracted through thrombus. Vascular device 150 comprises guide wire 152 having floppy distal end 153, sheath 154, and filter 156.

Sheath 154 comprises proximal section 158 having lumen 159, distal section 160 having lumen 161, and bridge section 162 disposed therebetween. Bridge section 162 may be a portion of proximal section 158 or of distal section 160 that has been cut away to provide a window through which filter 156 may expand. Alternatively, bridge section 162 may comprise a rod that connects the proximal section to the distal section.

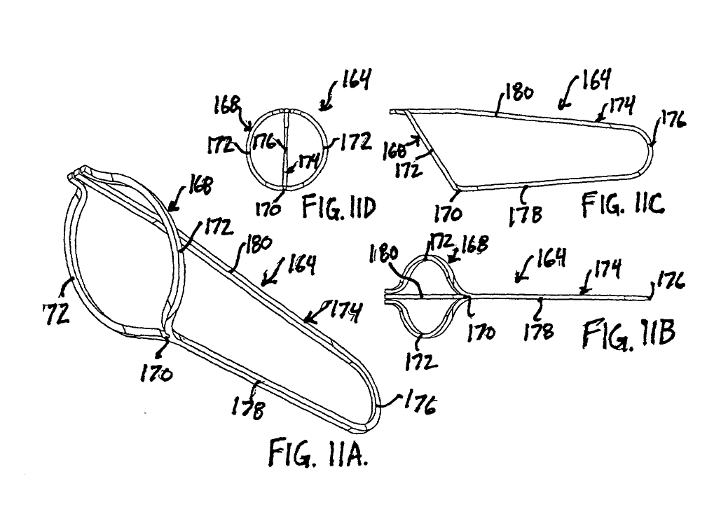

Filter 156 comprises filter frame 164, and blood permeable sac 166 attached thereto. Frame 164 is attached to guide wire 152 and is described in greater detail with respect to FIGS. 11A-11D. In use, filter 156 is disposed in a collapsed configuration during delivery within distal section 160 of sheath 154. Filter 156 then is expanded to the deployed configuration by retracting element 156 with respect to sheath 154 until the filter is disposed in bridge section 162 of the sheath. Frame 164 dynamically expands to the deployed configuration of FIG. 10. Vascular device 150 is then proximally retracted to draw filter 156 through thrombus and capture the thrombus in sac 166.

Referring now to FIGS. 11A-11D, in conjunction with FIG. 10, filter frame 164 is described in greater detail. Frame 164 comprises support hoop 168 having reduced-thickness articulation region 170 disposed between curved regions 172. Hoop 168 is coupled to arch support 174. Arch support 174 comprises hinge articulation region 176 disposed between first and second support struts 178 and 180, respectively. First strut 178 is coupled to articulation region 170, while second strut 180 is coupled to curved regions 172. Hinge section 176 and articulation region 170 are preferably formed of a superelastic material, for example, a nickel titanium alloy (nitinol) or spring tempered stainless steel. Guide wire 152 of FIG. 10 is preferably connected to first support strut 178, thereby providing filter frame 164 with the “proximal tilt” seen in FIG. 10.

When retracted through thrombus, arch support 174 provides structural stability that maintains frame 164 in the deployed configuration. Frame 164 may then be collapsed back to the delivery configuration by impinging distal section 160 of sheath 154 against hinge articulation 176. The hinge articulation deforms and advances second strut 180 with respect to first strut 178, thereby causing support hoop 168 to deform at articulation region 170 and collapse for retrieval within distal section 160.

In addition to its ability to maintain support hoop 168 in the deployed configuration, filter frame 164 provides blood permeable sac 166 with increased strength against breaking. It also prevents camming or stiction and allows easy movement back and forth of filter 156 within a patient's vessel without damaging the vessel walls.

With reference to FIG. 12, an alternative embodiment of the vascular device of FIG. 10 having a tension thread is described. Vascular device 190 comprises guide wire 192 having floppy distal end 193, sheath 194, and filter 196. Sheath 194 is similar to sheath 154 of FIG. 10 and comprises proximal section 198 having lumen 199, distal section 200 having lumen 201, and bridge section 202 disposed therebetween. Filter 196 comprises support hoop 204, blood permeable sac 206, and tension thread 208. Support hoop 204 is attached to guide wire 192. Likewise, sac 206 is attached to the guide wire along its length, and is also attached to support hoop 204. Support hoop 204 comprises articulation region 210 that allows support hoop 204 to collapse to the delivery configuration via sheathing from the distal side of the hoop.

Tension thread 208 is connected to support hoop 204 near articulation region 210, and is connected to guide wire 192 proximal of hoop 204. Thread 208 is dimensioned such that the thread is taut when hoop 204 is in the expanded deployed configuration of FIG. 12. When support hoop 204 is disposed in the delivery configuration within distal section 200 of sheath 194, the distance between where tension thread 208 is connected to support hoop 204 and where the tension thread is connected to guide wire 192 is shorter than when support hoop 204 is in the deployed configuration. Thus, tension thread 208 is lax in the delivery configuration. This is made possible by distal sheathing of filter 196.

When used in a thrombectomy application, such as the dual arrangement of FIGS. 5, filter 196 is expanded to the deployed configuration, and vascular device 190 is retracted proximally through thrombus. Taut tension thread 208 ensures that articulation region 210 does not articulate and that the mouth of support hoop 204 remains open while thrombus is captured in blood permeable sac 206.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

What is claimed is:

1. Apparatus suitable for filtering emboli or performing thrombectomy comprising:
an elongated member having a distal region;
a support hoop attached to the distal region, the support hoop having one or more articulation regions; and
a blood permeable sac affixed to the support hoop so that the hoop forms a mouth of the blood permeable sac.

2. The apparatus of claim 1 further comprising a thrombus removal element disposed proximal of the support hoop.

3. The apparatus of claim 2, wherein the thrombus removal element is adapted to be movable in a proximal direction independent of the elongated member.

4. The apparatus of claim 2, wherein the thrombus removal element comprises a second support hoop with blood permeable sac.

5. The apparatus of claim 4, wherein the second support hoop is slidably disposed on the elongated member.

6. The apparatus of claim 1, wherein the blood permeable sac comprises a plurality of pores dimensioned to allow passage of blood cells therethrough.

7. The apparatus of claim 1, wherein the support hoop comprises a super-elastic material.

8. The apparatus of claim 1, wherein the support hoop comprises a wire having a thickness that tapers to a minimum thickness at the one or more articulation regions.

9. The apparatus of claim 1, wherein the apparatus has a deployed state, wherein the hoop engages an interior wall of a patient's vessel, and a delivery state, wherein the apparatus has a contracted configuration to permit insertion within a delivery sheath.

10. The apparatus of claim 1, wherein the support hoop folds at the one or more articulation regions when the apparatus is contracted to the delivery state.

11. The apparatus of claim 2, wherein thrombus is excised or ablated by the thrombus removal element.

12. The apparatus of claim 9, wherein the mouth of the blood permeable sac is closed when the apparatus is in the contracted configuration, thereby preventing emboli from escaping from the blood permeable sac.

13. The apparatus of claim 10 wherein opposite sides of the support hoop close towards one another when the apparatus is contracted to its contracted configuration.

14. The apparatus of claim 1, wherein the support hoop comprises a radiopaque feature.

15. The apparatus of claim 1 further comprising:
a nose cone disposed on the distal region of the elongated member distal to the support hoop;
and a delivery sheath having a lumen for accepting the elongated member, support hoop and blood permeable sac.

16. The apparatus of claim 1, wherein the support hoop and blood permeable sac comprise a thrombus removal element.

17. The apparatus of claim 1 further comprising a spinner tube coaxially disposed about the distal region of the elongated member, the blood permeable sac coupled to the spinner tube.

18. The apparatus of claim 17, wherein the elongated member further comprises proximal and distal stops, the spinner tube disposed between the proximal and distal stops.

19. The apparatus of claim 17 further comprising a nose cone disposed on a distal end of the spinner tube.

20. The apparatus of claim 1, further comprising a delivery sheath for accepting the elongated member, support hoop and blood permeable sac, and wherein the delivery sheath further comprises a distal end having a positive locking feature.

21. The apparatus of claim 20, wherein the positive locking feature is configured to selectively maintain the support hoop in an expanded deployed configuration.

22. The apparatus of claim 21, wherein the positive locking feature comprises a wedge.

23. The apparatus of claim 1 further comprising a positive locking feature in communication with the support hoop, the positive locking feature configured to selectively maintain the support hoop in an expanded deployed configuration.

24. The apparatus of claim 1, further comprising a delivery sheath for accepting the elongated member, support hoop and blood permeable sac, and wherein the delivery sheath further comprises a distal section having the lumen, a proximal section having a second lumen, and a bridge section disposed between the proximal and distal sections.

25. The apparatus of claim 16, wherein the thrombus removal element further comprises an arch support coupled to the support hoop to form a filter frame.

26. The apparatus of claim 25, wherein the arch support comprises a hinge articulation region disposed between first and second support struts, the first and second support struts coupled to the support hoop.

27. The apparatus of claim 26, wherein the first support strut is further coupled to the elongated member.

28. The apparatus of claim 25 wherein the blood permeable sac is coupled to the filter frame.

29. The apparatus of claim 25 further comprising a delivery sheath having a distal section and a bridge section disposed proximal of the distal section, the distal section comprising a lumen for accepting the elongated member and thrombus removal element.

30. The apparatus of claim 16, wherein the thrombus removal element further comprises a tension thread, the tension thread coupled to the support hoop and to the elongated member proximal of the support hoop.

31. The apparatus of claim 30, wherein the tension thread is taut when the thrombectomy element is in an expanded deployed configuration and is lax when the thrombectomy element is in a collapsed delivery configuration.

32. The apparatus of claim 31, wherein the tension thread is configured to maintain the thrombectomy element in the deployed configuration during proximal retraction through thrombus.

33. The apparatus of claim 31 further comprising a delivery sheath having a distal section and a bridge section disposed proximal of the distal section, the distal section comprising a lumen for accepting the elongated member and thrombus removal element.

34. A method of trapping thrombus and/or filtering emboli during a medical procedure, the method comprising:
providing apparatus comprising an elongated member having a distal region and a filter, the filter comprising a support hoop having one or more articulation regions and coupled to the distal region of the elongated member, and a blood permeable sac affixed to the support hoop so that the hoop forms a mouth of the blood permeable sac;
positioning the apparatus in a contracted delivery state within a delivery sheath;
advancing the delivery sheath to a desired location within a patient's vessel; and
withdrawing the apparatus from the delivery sheath to expand the apparatus to a deployed state wherein the support hoop of the filter seals against the vessel wall.

35. The method of claim 34, wherein the filter comprises a thrombus removal element, the method further comprising moving the thrombus removal element relative to the vessel wall to excise thrombus from the vessel.

36. The method of claim 34, wherein advancing the delivery sheath to a desired location within a patient's vessel comprises advancing the delivery sheath through an interior lumen of a catheter so that a distal region of the delivery sheath is disposed distal to the lesion.

37. The method of claim 34 further comprising retracting the apparatus within the delivery sheath by folding the support hoop at the articulation region to close the mouth of the blood permeable sac.

38. The method of claim 34, wherein advancing the delivery sheath to the desired location within the patient's vessel comprises advancing the delivery sheath through tortuous anatomy to the desired location within the patient's vessel.

39. The method of claim 34 further comprising reversibly locking the support hoop in the deployed state.

40. The method of claim 34 further comprising applying tension to the support hoop in the deployed state with a tension thread.
